(19) United States
(12) Patent Application Publication
(10) Pub. No.: US 2006/0142764 A1
(43) Pub. Date: Jun. 29, 2006

(54) Intramedullary nail

(76) Inventor: Michael Zielsdorf, Vohl (DE)

Correspondence Address: LERNER GREENBERG STEMER LLP, P O BOX 2480, HOLLYWOOD, FL, 33022-2480, US

(21) Appl. No.: 11/318,197

(22) Filed: Dec. 23, 2005

(30) Foreign Application Priority Data
Dec. 23, 2004 (DE) 10 2004 063 396

(51) Int. Cl. A61F 2/30

(52) U.S. Cl. 606/62

(57) ABSTRACT

An intramedullary nail is provided for a closed reduction of a fracture of a tubular bone by insertion into a bore in the medullary cavity thereof. The intramedullary nail has a simple configuration, is practice-oriented, and can be used simply, quickly, safely and with only minimal use of intraoperative X-ray imaging, and without requiring interventions in undamaged areas of an extremity. The intramedullary nail has a nail core at a front area of which there is a head piece on which a start of a band is secured. The band is wound on around the nail core and, at its end in a rear area, is secured so that it can be locked in rotation around the nail core and which, in the event of a rotation counter to its direction of winding on, widens the radial contour of the intramedullary nail.

### BACKGROUND OF THE INVENTION

### Field of the Invention

The invention relates to an intramedullary nail in particular for closed reduction of a fracture of a tubular bone by insertion into a bore in the medullary cavity thereof.

A device for stabilizing tubular bone fractures and joints is known from German patent DE 38 35 682. The device contains, as stabilizing elements, helical springs which are interwound in one or several turns and which are partially wound around a core in the form of an elastically deformable wire or a bundle of elastically deformable wires. Such a helical spring can be provided at its ends with a device for fixing to the bone.

A disadvantage of the known device is the uncertainty of the position of the helical springs, and also the impossibility of preventing rotation and the impossibility of fixing individual fragments in the area of a fracture, because the spring characteristics of the helical springs cannot be avoided there.

A nail system for intramedullary osteosynthesis of shaft fractures of all kinds is also known, from published, non-prosecuted German patent application DE 197 31 298 A1, which system contains a tubular outer body with openings out of which hooks extend, and, by activation of an inner body, the hooks can be pivoted outward, as a result of which the hooks are intended to be able to bear on the inner wall of a tubular bone.

The nail system is extremely complex and has a very large number of component parts. It can in principle only permit punctiform contacts with the inner surface of a tubular bone, and it can permit these only within a narrow peripheral area identical along its length.

Simpler intramedullary nails for locking are known, but their placement during an intervention can be controlled only by intraoperative X-ray imaging which, because of its duration, not only places a burden on the patient but in particular also on the operating personnel.

Moreover, simple intramedullary nails of this kind have to be secured in the bone by transverse screws, for which purpose, particularly in the case of long femoral bones, but also in the case of long humeral bones and tibial bones, elaborate targeting devices are needed in order for the transverse screws to be able to be passed at the exact point through the skin, with the least possible damage to the latter. These surgical wounds additionally made in an extremity have to be carefully managed after an operation, but also after surgical removal of the transverse screws, which fact places an additional burden on the patient.

### SUMMARY OF THE INVENTION

It is accordingly an object of the invention to provide an intramedullary nail which overcomes the above-mentioned disadvantages of the prior art devices of this general type, which has a simple construction, is practice-oriented, and can be used simply, quickly, safely and with only minimal use of intraoperative X-ray imaging, and without requiring interventions in undamaged areas of an extremity.

With the foregoing and other objects in view there is provided, in accordance with the invention, an intramedullary nail for closed reduction of a fracture of a tubular bone by insertion into a bore in a medullary cavity of the tubular bone. The intramedullary nail contains a nail core having a front area and a rear area, a head piece disposed at the front area of the nail core, and a band having a starting end secured to the head piece. The band is wound on around the nail core. The band has a rear end secured to the rear area of the nail core so that the rear end is locked in rotation around the nail core and the band, in an event of a rotation counter to a direction of winding on of the band, widens a radial contour of the intramedullary nail.

The intramedullary nail, which is suitable in particular for closed reduction of a fracture of a tubular bone by insertion into a bore in the medullary cavity thereof, is characterized in that it has a nail core at the front area of which the start of an outer band is secured. The band is wound on around the nail core and, at its end in the rear area, is secured so that it can be locked in rotation around the nail core. The radial contour of the intramedullary nail widens in the event of a rotation counter to its direction of winding on. This has the advantageous result that the outer face of the band can bear on the inner face of a through-bore or directly on the inner face of the medullary cavity of a tubular bone, and, by further turning counter to the direction of winding on, a radially acting force can be generated with which the individual parts of a bone can be clamped in an optimally aligned and reduced state, by which it is possible to achieve the best possible healing result.

The technical structure of such an intramedullary nail is extremely simple, can be easily understood by any physician providing emergency treatment, and is thus also safe in correct use. It is in this case possible to entirely dispense with distal fixing screws for the nail, with the result that postoperative management of incision wounds is no longer necessary in this area.

The clamping force of the band ensures a strong lateral cortical support and, therefore, optimal stabilization of a fracture, and this also leads to a more rapid course of healing of the fracture. Disruptions to the healing of the fracture as a result of inadequate fracture fixation can be completely eliminated using the intramedullary nail according to the invention. However, longitudinally divided bone fractures or those situated very close to a joint cannot be treated with the intramedullary nail according to the invention. In order to remove the intramedullary nail, the bands are again wound more strongly on around the nail core, as a result of which the external diameter of the intramedullary nail decreases again, so that it can be withdrawn without any problem from the bore or the medullary cavity.

A particularly advantageous embodiment is one in which the start of a further, inner band is secured in the front area of the intramedullary nail, which further band is wound on around the nail core under the outer band and its end, in the rear area of the intramedullary nail, is likewise secured so that it can be locked in rotation around the nail core. In the event of a rotation counter to its direction of winding on, such an inner band increases the external diameter of the intramedullary nail and bears with its outer face on the inner face of the outer band and thus stabilizes the band and, consequently, the entire intramedullary nail. This construction permits a high degree of stiffness, which can correspond approximately to that of a thin tube.

Since the band is made from a resiliently elastic material, for example from surgical steel, titanium or biocompatible plastic, it is possible to optimally adapt the desired properties of the intramedullary nail in respect of the required contact force and also the nature and course of the deformation, advantageously starting from the front area of the intramedullary nail, via the narrowed part of a medullary space, to the once again widening end of the medullary space.

According to other advantageous embodiments of the invention, several inner or outer bands can also be wound on around the nail core in the same direction or above all in opposite directions and in a single turn or multiple turns. In the case of a version with a single turn of the inner bands, these are wound on directly around the core, and in the case of an embodiment with a single turn of the outer bands, these are wound on around the inner bands.

This wealth of variation results in that it is possible to provide an optimally adapted intramedullary nail for the most common types of fractures of bones of any desired size, since the required strength can be set at almost any desired level. It is particularly advantageous that the radial outer surfaces of the inner band and the radial inner surfaces of the outer band are configured as mutual function surfaces, such that the common contact areas of the bands form stabilization points of an intramedullary nail inserted into a through-bore of a bone and clamped, the stabilization points being established in the form of a frictional connection. The stability of the intramedullary nail is further increased by this measure.

In a preferred embodiment of the invention, the bands in the unworked state are pretensioned and, around the nail core in the area of their rear ends, are locked against one another or against the nail core via a clamping and closing mechanism, so that, after unlocking, they automatically untwist counter to the sense of rotation of the winding and thus increase the external diameter of the intramedullary nail either uniformly or, advantageously however, in such a way that the outer band bears on the inner contours of the medullary space of a tubular bone. Such a function can, for example, be produced by the fact that the inner band and/or the outer band are configured with a different width or thickness in different areas of the nail core, or the inner band and/or the outer band are elastically pretensioned to different extents in different areas of the nail core. It is also conceivable that, for example, the outer band, along its entire length, or at least along the first ⅔ of its length as seen from the needle tip, has a continuously changing cross section, in particular an increasing width, in order to satisfy the required function.

An advantageous construction of this kind also results in that, even in the case of bands that are not pretensioned, that the outer band, when being turned counter to the direction of rotation, bears initially in the area of the head piece of the intramedullary nail on the inner contour of a bone and, only upon further turning, bears initially in the middle area of the narrowed part of the medullary space of a tubular bone and thereafter, in the end area of the intramedullary nail, on the once again widening area of the medullary space.

To guarantee this function, the intramedullary nail is equipped at its rear end with a clamping and closing mechanism containing at least an inner bushing and an outer bushing which have mutual locks or locking surfaces, for example self-locking conical seats. The inner band is secured on the inner bushing which is rotatable about the nail core, and the outer band is secured on the outer bushing which is likewise rotatable about the nail core.

To fit the intramedullary nail in place, it is equipped with a detachable assembly aid which has actuating devices for the clamping and closing mechanism.

At its rear area, the intramedullary nail advantageously has contact surfaces for the application of an adapter as a further mechanical device for stabilization of bone fractures, as is known for example for a fracture of the neck of the femur.

The end of the nail core is advantageously provided with a thread on which the inner bushing of the clamping and closing mechanism is screwed, thereby guaranteeing that, when the inner bushing and outer bushing are mutually locked, a defined position of the clamping and closing mechanism relative to the nail core is obtained.

In a further advantageous embodiment of the subject matter of the invention, the nail core is hollow and is traversed by a sleeve and/or shaft, so that the start of an inner and/or outer band can be secured at the front area of the head piece of the intramedullary nail, in which case, from the rear end of the intramedullary nail, the latter can be unwound counter to its direction of rotation via a suitably configured clamping and closing mechanism and stretched.

One inventive development of the intramedullary nail contains two outer bands, for example, one of which can be turned from the front area of the intramedullary nail and the other from the rear area of the intramedullary nail, so as to permit optimal adaptation of the band to the medullary space widening in a trumpet shape in both directions from a narrowed part of the medullary space and, consequently, an optimal lateral cortical support.

Depending on the specific requirements, the intramedullary nail can be straight or bent or can also be plastically ductile in accordance with the required spatial form.

Moreover, the nail core of an intramedullary nail can advantageously be in several parts, such that these parts are able to turn relative to one another. This very considerably widens the structural possibilities.

In another very advantageous embodiment of the invention, two bands wound on opposite to one another are connected to one another at their front ends, or a band is configured in one piece and, starting from its front end, is wound on in opposite directions around the nail core, such that the nail core can be made short and extends only to a partial extent in the direction of the front end of the intramedullary nail. In an extreme version of this embodiment, the nail core is even reduced to just the clamping and closing mechanism, as a result of which, in the state for insertion, a stable rigid lattice structure remains as the intramedullary nail, which leads to an enormous saving in material and weight and also to a very inexpensive variant.

Other features which are considered as characteristic for the invention are set forth in the appended claims.

Although the invention is illustrated and described herein as embodied in an intramedullary nail, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE DRAWINGS

FIG. 1 is a diagrammatic, top plan view of an intramedullary nail according to the invention;

FIG. 2 is a diagrammatic, partial sectional view of a clamping and closing mechanism;

FIG. 3 is a diagrammatic, sectional view through a wrench toothing of the clamping and closing mechanism;

FIG. 4 is a diagrammatic, sectional view through an inner locking of an inner bushing and an outer bushing; and

FIG. 5 is a diagrammatic, top plan view of a second embodiment of the intramedullary nail with greatly reduced nail core and according to the invention.

### DESCRIPTION OF THE PREFERRED EMBODIMENTS

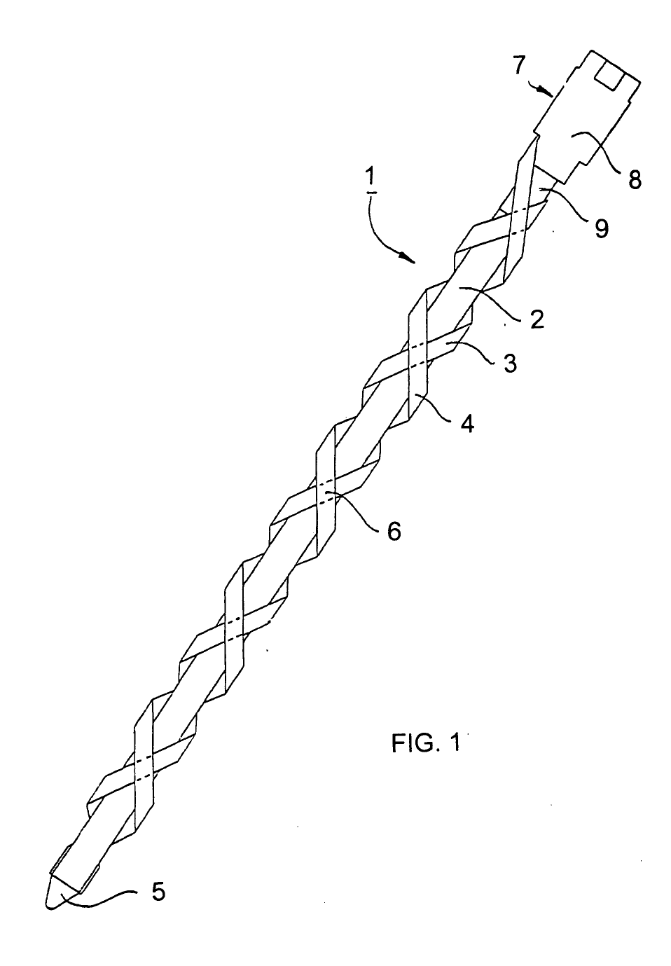

Referring now to the figures of the drawing in detail and first, particularly, to FIG. 1 thereof, there is shown an intramedullary nail 1. The intramedullary nail 1 contains a nail core 2 around which an inner band 3 is wound on starting from a head piece 5, and around which an outer band 4 is wound on in an opposite direction to the inner band 3, the front ends of the bands 3, 4 being applied in a fixed position on the head piece 5, for example by a laser welding method.

In a rear area of the intramedullary nail 1, the ends of the bands 3, 4 are disposed on an outer bushing 8 and an inner bushing 9, respectively, of a clamping and closing mechanism 7 and are welded there, for example once again by a laser. The bands 3, 4 wound on in opposite directions overlap one another at contact areas 6, the surfaces of the bands 3, 4 directed toward one another being configured in such a way that a frictional connection is established there which stabilizes the entire intramedullary nail 1.

The outer bushing 8 and the inner bushing 9 have a mutual locking configuration and are secured via a thread 10 on the end of the nail core 2 and in the inner bushing 9 so as to be able to turn on the threading 10.

The embodiment shown only by way of example in FIG. 1 contains, in a practice-oriented embodiment, a much greater number of windings of the inner band 3 and also of the outer band 4, so that a considerably greater number of common contact areas 6 between the bands 3, 4 is obtained, consequently leading to a much increased stiffness of the intramedullary nail 1 and to an associated optimized fixing of a fracture.

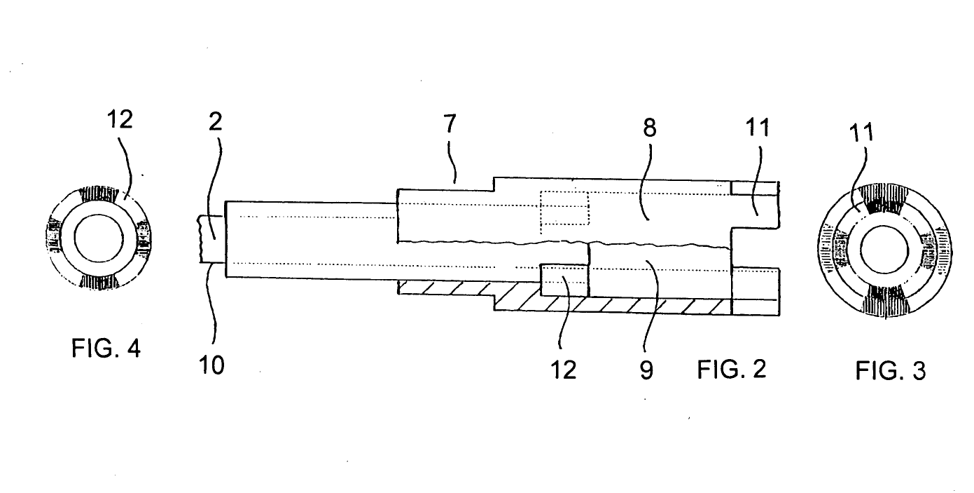

FIGS. 2 through 4 also show only illustrative examples of the function of the clamping and closing mechanism 7 and its wrench toothing 11 for attachment of a non-illustrated assembly aid, and a section through an inner locking 12 of the inner bushing 9 and the outer bushing 8.

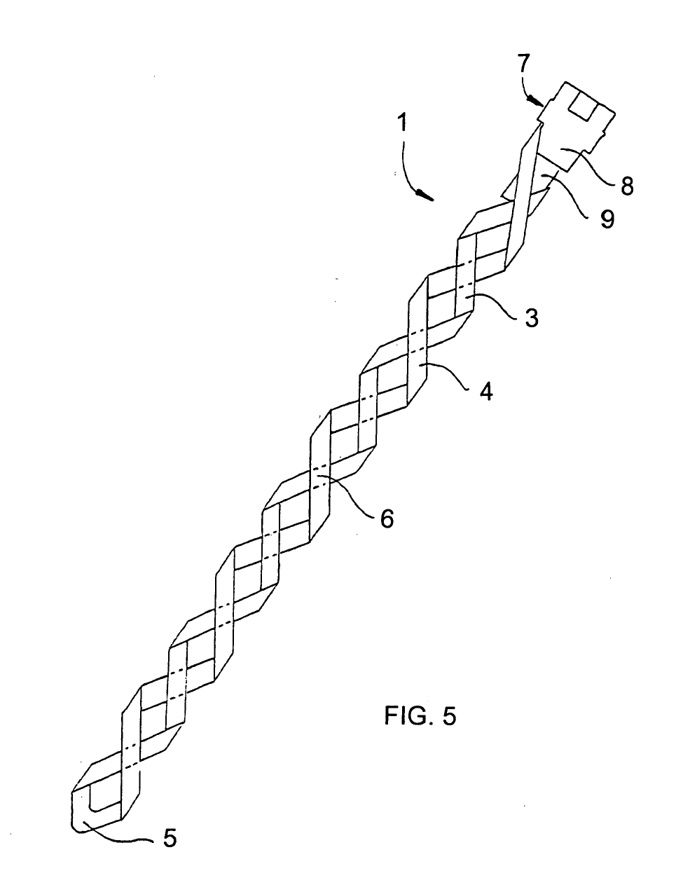

The embodiment shown in FIG. 5, likewise only by way of example, also contains, in a practice-oriented embodiment,a much greater number of windings and, in this case, only a single band, which is wound on in opposite directions starting from the tip, configured as the head piece 5, and whose inner windings form the inner band 3, while the outer windings form the outer band 4. The actual nail core 2 is in this case completely reduced to just the clamping and closing mechanism 7 and replaced by a free air space. This version too permits clamping of the intramedullary nail free from forces from outside, as is also the case in all the other aforementioned versions, without mechanical restoring forces having to be taken up by a bone or having to be introduced into the latter.

This application claims the priority, under 35 U.S.C. § 119, of German patent application DE 10 2004 063 396, filed Dec. 23, 2004; the entire disclosure of the prior application is herewith incorporated by reference.

I claim:

1. An intramedullary nail for closed reduction of a fracture of a tubular bone by insertion into a bore in a medullary cavity of the tubular bone, the intramedullary nail comprising:
a nail core having a front area and a rear area;
a head piece disposed at said front area of said nail core; and
a band having a starting end secured to said head piece, said band wound on around said nail core, said band having a rear end secured to said rear area of said nail core so that said rear end being locked in rotation around said nail core and said band, in an event of a rotation counter to a direction of winding on of said band, widens a radial contour of the intramedullary nail.

2. The intramedullary nail according to claim 1, further comprising an inner band with a starting end secured in said front area of said nail core, said inner band being wound on around said nail core under said band, said inner band having a rear end secured to said rear area so that said inner band is locked in rotation around said nail core and said inner band, in an event of a rotation counter to a direction of winding on said nail core, bears on said band being an outer band in a region of common contact areas with said outer band and stabilizes the intramedullary nail.

3. The intramedullary nail according to claim 2, wherein said inner band and said outer band are wound on around said nail core in a same direction.

4. The intramedullary nail according to claim 2, wherein said inner band and said outer band are wound on around said nail core in a same direction of winding in multiple turns.

5. The intramedullary nail according to claim 2, wherein said inner band and said outer band have radial outer surfaces configured as mutual function surfaces which, at said common contact areas, form stabilization points of the intramedullary nail inserted into a through-bore of the tubular bone and clamped, said stabilization points being established in a form of a frictional connection.

6. The intramedullary nail according to claim 2, wherein at least one of said outer band and said inner band in an uninserted state is secured with pretensioning on said nail core.

7. The intramedullary nail according to claim 2, wherein said outer band when unwound counter to said direction of winding is configured such that said outer band bears on inner contours of a medullary space of the tubular bone.

8. The intramedullary nail according to claim 2, wherein at least one of said inner band and said outer band have a different width and/or a different thickness in different areas along said nail core.

9. The intramedullary nail according to claim 2, wherein said outer band is configured such that, when being turned counter to said direction of winding, said outer band bears initially in an area of said head piece of the intramedullary nail on an inner contour of the tubular bone and, upon further turning, then bears initially in a middle area of the tubular bone and thereafter in an end area of the intramedullary nail.

10. The intramedullary nail according to claim 2, further comprising a clamping and closing mechanism, said clamping and closing mechanism including:
an inner bushing rotatably mounted on said nail core, said inner band being secured to said inner bushing; and
an outer bushing rotatably mounted on said nail core, said outer band secured to said outer bushing, both of said inner and outer bushings having securing elements selected from the group consisting of locks and locking surfaces, said securing elements being locked or clamped against one another and/or against said nail core.

11. The intramedullary nail according to claim 10, further comprising a detachable assembly aid having actuating devices for engaging said clamping and closing mechanism.

12. The intramedullary nail according to claim 1, wherein at least one of said front area and said rear area has contact surfaces for an application of further mechanical devices for stabilization of bone fractures.

13. The intramedullary nail according to claim 10, wherein said nail core at said rear area has a threading formed thereon, said inner bushing being screwed on said threading such that said inner bushing can turn.

14. The intramedullary nail according to claim 10, wherein:
said nail core has a hollow interior formed therein and an element selected from the group consisting of a shaft and a sleeve traversing said hollow interior, said element having a front end securing said starting end of said inner band and said outer band; and
said clamping and closing mechanism is disposed at said rear area of said nail core.

15. The intramedullary nail according to claim 14, wherein said outer band is formed of a first outer band and a second outer band, said first outer band having a starting end secured to said front area of said nail core, said second outer band is secured at said rear area of said nail core, and both said first and second outer bands run toward one another and extend in a same plane in respective clearances of the other band as far as a respective other end of said nail core.

16. The intramedullary nail according to claim 1, wherein the intramedullary nail is straight or bent.

17. The intramedullary nail according to claim 1, wherein said nail core is formed in several parts and said parts can turn relative to one another.

18. The intramedullary nail according to claim 10, wherein said inner and outer bands are wound on opposite to one another and are connected to one another at said starting ends.

19. The intramedullary nail according to claim 2, wherein said inner band and said outer band are wound on around said nail core in opposite directions.

20. The intramedullary nail according to claim 2, wherein said rear end of said outer band is locked against said rear end of said inner band.

21. The intramedullary nail according to claim 2, wherein said inner and outer bands are configured as a one piece band having a front end, and starting from said front end, said inner and outer bands are wound on in opposite directions around said nail core, and in that said nail core is made short and extends only to a partial extent in a direction of a front end of the intramedullary nail.

22. An intramedullary nail for closed reduction of a fracture of a tubular bone by insertion into a bore in a medullary cavity of the tubular bone, the intramedullary nail comprising:
a nail core formed as a clamping and closing mechanism having an inner bushing and an outer bushing; and
a single band wound in opposite directions starting from a tip, configured as a head piece, said single band having inner windings forming an inner band and outer windings forming an outer band, said inner band secured to said inner bushing and said outer band secured to said outer bushing, and in an event of a rotation counter to a direction of winding on of said outer band, said single band widens a radial contour of the intramedullary nail.
